(19) United States
(12) Patent Application Publication
(10) Pub. No.: US 2003/0086913 A1
(43) Pub. Date: May 8, 2003

(54) Gene transfer vector composition

(75) Inventors: Bryan Butman, Walkersville, MD (US); Stephen Morris, Rockville, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: GenVec, Inc., Gaithersburg, MD (US)

(21) Appl. No.: 10/003,624

(22) Filed: Nov. 2, 2001

(51) Int. Cl. A61K 48/00; C12N 15/86

(52) U.S. Cl. 424/93.21; 600/1; 435/456

(57) ABSTRACT

The invention provides a composition comprising a gene transfer vector which comprises a nucleic acid sequence encoding a protein and a carrier therefore. The composition is characterized by a relatively high ratio of the gene transfer vector to the protein in the composition.

### FIELD OF THE INVENTION

This invention pertains to a gene transfer vector composition.

### BACKGROUND OF THE INVENTION

The significant progress of human genetics and gene therapy research presents researchers with the opportunity to use gene therapy as a treatment modality for a number of disease states. A nucleic acid sequence encoding a protein of interest is transferred to a host cell (e.g., in a person) by way of a gene transfer vector. Once the nucleic acid sequence is in the cell, it is expressed to produce the protein, which desirably is a therapeutic protein. Eukaryotic viruses, particularly replication-deficient viruses, are the vectors of choice for many gene therapy strategies. Prior to human administration, viral gene transfer vectors are purified from the cells in which they are produced; however, the residual presence of the protein encoded by the nucleic acid sequence in the gene transfer vector composition, which protein is expressed in the cells in which the gene transfer vector is produced, can result in a patient's being exposed to the protein prior to the expression of the nucleic acid sequence in the host cells of the patient.

Many of the therapeutic proteins currently under investigation in pre-clinical or clinical trials do not exhibit harmful side effects when present in a patient prior to expression of the nucleic acid sequence in the host cell of the patient. Some proteins, however, such as tumor necrosis factor (TNF), cause adverse effects when exposed to non-target tissues. Inflammation, irritation, and allergic reactions are some of the possible side effects of exposure to a protein upon administration of a gene transfer vector composition containing the protein.

In view of the problems associated with gene transfer vector compositions containing the protein encoded by the nucleic acid sequence of interest in the gene transfer vector, there remains a need for a gene transfer vector composition of improved purity. The invention provides such a composition. These and other advantages of the invention, as well as additional inventive features, will be apparent from the description of the invention provided herein.

### BRIEF SUMMARY OF THE INVENTION

The invention provides a composition comprising (a) about 1×10⁵ or more particle units of a gene transfer vector comprising a nucleic acid sequence encoding a protein and (b) a carrier therefor. The ratio of the gene transfer vector to the protein in the composition is about 6.4×10⁹ or more particle units gene transfer vector:1 picogram protein.

### DETAILED DESCRIPTION OF THE INVENTION

The invention provides a gene transfer vector composition. The composition comprises (a) about 1×10⁵ or more particle units of a gene transfer vector comprising a nucleic acid sequence encoding a protein and (b) a carrier therefor. The ratio of the gene transfer vector to the protein produced by expression of the nucleic acid sequence of the gene transfer vector in the composition is about 6.4×10⁹ or more particle units of gene transfer vector:1 picogram of protein. The gene transfer vector can be any suitable gene transfer vector. Examples of suitable gene transfer vectors include plasmids, liposomes, molecular conjugates (e.g., transferrin), and viruses. Preferably, the gene transfer vector is a viral vector. Suitable viral vectors include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Retrovirus is an RNA virus capable of infecting a wide variety of host cells. Upon infection, the retroviral genome integrates into the genome of its host cell and is replicated along with host cell DNA, thereby constantly producing viral RNA and any nucleic acid sequence incorporated into the retroviral genome. As such, long-term expression of a therapeutic factor(s) is achievable when using retrovirus. Retroviruses contemplated for use in gene therapy are relatively non-pathogenic, although pathogenic retroviruses exist. When employing pathogenic retroviruses, e.g., human immunodeficiency virus (HIV) or human T-cell lymphotrophic viruses (HTLV), care must be taken in altering the viral genome to eliminate toxicity to the host. A retroviral vector additionally can be manipulated to render the virus replication-deficient. As such, retroviral vectors are considered particularly useful for stable gene transfer in vivo. Lentiviral vectors, such as HIV-based vectors, are exemplary of retroviral vectors used for gene delivery. Unlike other retroviruses, HIV-based vectors are known to incorporate their passenger genes into non-dividing cells and, therefore, can be of use in treating persistent forms of disease.

An HSV-based viral vector is suitable for use as a gene transfer vector to introduce a nucleic acid into numerous cell types. The mature HSV virion consists of an enveloped icosahedral capsid with a viral genome consisting of a linear double-stranded DNA molecule that is 152 kb. Most replication-deficient HSV vectors contain a deletion to remove one or more intermediate-early genes to prevent replication. Advantages of the HSV vector are its ability to enter a latent stage that can result in long-term DNA expression and its large viral DNA genome that can accommodate exogenous DNA inserts of up to 25 kb. Of course, the ability of HSV to promote long-term production of exogenous protein is potentially disadvantageous in terms of short-term treatment regimens. However, one of ordinary skill in the art has the requisite understanding to determine the appropriate vector for a particular situation. HSV-based vectors are described in, for example, U.S. Pat. Nos. 5,837,532, 5,846,782, 5,849,572, and 5,804,413, and International Patent Applications WO 91/02788, WO 96/04394, WO 98/15637, and WO 99/06583.

AAV vectors are viral vectors of particular interest for use in gene therapy protocols. AAV is a DNA virus, which is not known to cause human disease. The AAV genome is comprised of two genes, rep and cap, flanked by inverted terminal repeats (ITRs), which contain recognition signals for DNA replication and packaging of the virus. AAV requires co-infection with a helper virus (i.e., an adenovirus or a herpes simplex virus), or expression of helper genes, for efficient replication. AAV can be propagated in a wide array of host cells including human, simian, and rodent cells, depending on the helper virus employed. An AAV vector used for administration of a nucleic acid sequence typically has approximately 96% of the parental genome deleted, such that only the ITRs remain. This eliminates immunologic or toxic side effects due to expression of viral genes. If desired, the AAV rep protein can be co-administered with the AAV vector to enable integration of the AAV vector into the host cell genome. Host cells comprising an integrated AAV genome show no change in cell growth or morphology (see, e.g., U.S. Pat. No. 4,797,368). As such, prolonged expression of therapeutic factors from AAV vectors can be useful in treating persistent and chronic diseases.

The viral vector is most preferably an adenoviral vector. Adenovirus (Ad) is a 36 kb double-stranded DNA virus that efficiently transfers DNA in vivo to a variety of different target cell types. The adenoviral vector can be produced in high titers and can efficiently transfer DNA to replicating and non-replicating cells. The adenoviral vector genome can be generated using any species, strain, subtype, mixture of species, strains, or subtypes, or chimeric adenovirus as the source of vector DNA. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Given that the human adenovirus serotype 5 (Ad5) genome has been completely sequenced, the adenoviral vector of the invention is described herein with respect to the Ad5 serotype. The adenoviral vector can be any adenoviral vector capable of growth in a cell, which is in some significant part (although not necessarily substantially) derived from or based upon the genome of an adenovirus. The adenoviral vector can be based on the genome of any suitable wild-type adenovirus. Preferably, the adenoviral vector is derived from the genome of a wild-type adenovirus of group C, especially of serotype 2 or 5. Adenoviral vectors are well known in the art and are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, and WO 00/00628, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996).

Preferably, the adenoviral vector is replication-deficient. By “replication-deficient” is meant that the adenoviral vector comprises a genome that lacks at least one replication-essential gene function. A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (i.e., propagation) of a replication-deficient adenoviral vector. Replication-essential gene functions are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA I and/or VA-RNA II). Preferably, the replication-deficient adenoviral vector comprises an adenoviral genome deficient in at least one replication-essential gene function of one or more regions of an adenoviral genome (e.g., two or more regions of an adenoviral genome so as to result in a multiply replication-deficient adenoviral vector). The one or more regions of the adenoviral genome are preferably selected from the group consisting of the E1, E2, and E4 regions. More preferably, the replication-deficient adenoviral vector comprises a deficiency in at least one replication-essential gene function of the E1 region (denoted an E1-deficient adenoviral vector), particularly a deficiency in a replication-essential gene function of each of the adenoviral E1A region and the adenoviral E1B region. In addition to such a deficiency in the E1 region, the recombinant adenovirus also can have a mutation in the major late promoter (MLP), as discussed in International Patent Application WO 00/00628. More preferably, the vector is deficient in at least one replication-essential gene function of the E1 region and at least part of the nonessential E3 region (e.g., an Xba I deletion of the E3 region) (denoted an E1/E3-deficient adenoviral vector).

Preferably, the adenoviral vector is “multiply deficient,” meaning that the adenoviral vector is deficient in one or more gene functions required for viral replication in each of two or more regions of the adenoviral genome. For example, the aforementioned E1-deficient or E1/E3-deficient adenoviral vector can be further deficient in at least one replication-essential gene function of the E4 region (denoted an E1/E4-deficient adenoviral vector). An adenoviral vector deleted of the entire E4 region can elicit a lower host immune response.

Alternatively, the adenoviral vector lacks replication-essential gene functions in all or part of the E1 region and all or part of the E2 region (denoted an E1/E2-deficient adenoviral vector). Adenoviral vectors lacking replication-essential gene functions in all or part of the E1 region, all or part of the E2 region, and all or part of the E3 region also are contemplated herein. If the adenoviral vector of the invention is deficient in a replication-essential gene function of the E2A region, the vector preferably does not comprise a complete deletion of the E2A region, which is less than about 230 base pairs in length. Generally, the E2A region of the adenovirus codes for a DBP (DNA binding protein), a polypeptide required for DNA replication. DBP is composed of 473 to 529 amino acids depending on the viral serotype. It is believed that DBP is an asymmetric protein that exists as a prolate ellipsoid consisting of a globular Ct with an extended Nt domain. Studies indicate that the Ct domain is responsible for DBP's ability to bind to nucleic acids, bind to zinc, and function in DNA synthesis at the level of DNA chain elongation. However, the Nt domain is believed to function in late gene expression at both transcriptional and post-transcriptional levels, is responsible for efficient nuclear localization of the protein, and also may be involved in enhancement of its own expression. Deletions in the Nt domain between amino acids 2 to 38 have indicated that this region is important for DBP function (Brough et al., Virology, 196, 269-281 (1993)). While deletions in the E2A region coding for the Ct region of the DBP have no effect on viral replication, deletions in the E2A region which code for amino acids 2 to 38 of the Nt domain of the DBP impair viral replication. It is preferable that the multiply replication-deficient adenoviral vector contain this portion of the E2A region of the adenoviral genome. In particular, for example, the desired portion of the E2A region to be retained is that portion of the E2A region of the adenoviral genome which is defined by the 5′ end of the E2A region, specifically positions Ad5(23816) to Ad5(24032) of the E2A region of the adenoviral genome of serotype Ad5.

The adenoviral vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case it is preferred that at least either the viral inverted terminal repeats (ITRs) and one or more promoters or the viral ITRs and a packaging signal are left intact (i.e., an adenoviral amplicon). The larger the region of the adenoviral genome that is removed, the larger the piece of exogenous nucleic acid sequence that can be inserted into the genome. For example, given that the adenoviral genome is 36 kb, by leaving the viral ITRs and one or more promoters intact, the exogenous insert capacity of the adenovirus is approximately 35 kb. Alternatively, a multiply deficient adenoviral vector that contains only an ITR and a packaging signal effectively allows insertion of an exogenous nucleic acid sequence of approximately 37-38 kb. Of course, the inclusion of a spacer element in any or all of the deficient adenoviral regions will decrease the capacity of the adenoviral vector for large inserts. Suitable replication-deficient adenoviral vectors, including multiply deficient adenoviral vectors, are disclosed in U.S. Pat. Nos. 5,851,806 and 5,994,106 and International Patent Applications WO 95/34671 and WO 97/21826. An especially preferred adenoviral vector for use in the present inventive method is that described in International Patent Application PCT/US01/20536.

It should be appreciated that the deletion of different regions of the adenoviral vector can alter the immune response of the mammal. In particular, the deletion of different regions can reduce the inflammatory response generated by the adenoviral vector. Furthermore, the adenoviral vector's coat protein can be modified so as to decrease the adenoviral vector's ability or inability to be recognized by a neutralizing antibody directed against the wild-type coat protein, as described in International Patent Application WO 98/40509.

The adenoviral vector, when multiply replication-deficient, especially in replication-essential gene functions of the E1 and E4 regions, preferably includes a spacer element to provide viral growth in a complementing cell line similar to that achieved by singly replication deficient adenoviral vectors, particularly an adenoviral vector comprising a deficiency in the E1 region. The spacer element can contain any sequence or sequences which are of the desired length. The spacer element sequence can be coding or non-coding and native or non-native with respect to the adenoviral genome, but does not restore the replication-essential function to the deficient region. In the absence of a spacer, production of fiber protein and/or viral growth of the multiply replication-deficient adenoviral vector is reduced by comparison to that of a singly replication-deficient adenoviral vector. However, inclusion of the spacer in at least one of the deficient adenoviral regions, preferably the E4 region, can counteract this decrease in fiber protein production and viral growth. The use of a spacer in an adenoviral vector is described in U.S. Pat. No. 5,851,806.

Construction of adenoviral vectors is well understood in the art. Adenoviral vectors can be constructed and/or purified using the methods set forth, for example, in U.S. Pat. No. 5,965,358 and International Patent Applications WO 98/56937, WO 99/15686, and WO 99/54441. The production of adenoviral gene transfer vectors is well known in the art, and involves using standard molecular biological techniques such as those described in, for example, Sambrook et al., supra, Watson et al., supra, Ausubel et al., supra, and in several of the other references mentioned herein.

Replication-deficient adenoviral vectors are typically produced in complementing cell lines that provide gene functions not present in the replication-deficient adenoviral vectors, but required for viral propagation, at appropriate levels in order to generate high titers of viral vector stock. A preferred cell line complements for at least one and preferably all replication-essential gene functions not present in a replication-deficient adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons, which comprise minimal adenoviral sequences, such as only inverted terminal repeats (ITRs) and the packaging signal or only ITRs and an adenoviral promoter). Most preferably, the complementing cell line complements for a deficiency in at least one replication-essential gene function (e.g., two or more replication-essential gene functions) of the E1 region of the adenoviral genome, particularly a deficiency in a replication-essential gene function of each of the E1A and E1B regions. In addition, the complementing cell line can complement for a deficiency in at least one replication-essential gene function of the E2 (particularly as concerns the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome. The cell line preferably is further characterized in that it contains the complementing genes in a non-overlapping fashion with the adenoviral vector, which minimizes, and practically eliminates, the possibility of the vector genome recombining with the cellular DNA. Accordingly, the presence of replication competent adenoviruses (RCA) is minimized if not avoided in the vector stock, which, therefore, is suitable for certain therapeutic purposes, especially gene therapy purposes. The lack of RCA in the vector stock avoids the replication of the adenoviral vector in non-complementing cells. The construction of complementing cell lines involves standard molecular biology and cell culture techniques, such as those described by Sambrook et al., supra, and Ausubel et al., supra. Complementing cell lines for producing the gene transfer vector (e.g., adenoviral vector) include, but are not limited to, 293 cells (described in, e.g., Graham et al., J. Gen. Virol., 36, 59-72 (1977)), PER.C6 cells (described in, e.g., International Patent Application WO 97/00326, and U.S. Pat. Nos. 5,994,128 and 6,033,908), and 293-ORF6 cells (described in, e.g., International Patent Application WO 95/34671 and Brough et al., J. Virol., 71, 9206-9213 (1997)).

The gene transfer vector comprises a nucleic acid sequence encoding a protein (i.e., one or more nucleic acid sequences encoding one or more proteins). The nucleic acid sequence encoding the protein can be obtained from any source, e.g., isolated from nature, synthetically generated, isolated from a genetically engineered organism, and the like. An ordinarily skilled artisan will appreciate that any type of nucleic acid sequence (e.g., DNA, RNA, and cDNA) that can be inserted into a gene transfer vector can be used in connection with the invention. Whatever type of nucleic acid sequence is used, the nucleic acid sequence preferably encodes a secreted protein. By “secreted protein” is meant any peptide, polypeptide, or portion thereof, which is released by a cell into the extracellular environment. When the gene transfer vector is a replication-deficient adenovirus, the nucleic acid sequence encoding the protein is preferably located in the E1 region of the adenoviral genome. The insertion of a nucleic acid sequence into the adenoviral genome (e.g., the E1 region of the adenoviral genome) can be facilitated by known methods, for example, by the introduction of a unique restriction site at a given position of the adenoviral genome.

The nucleic acid sequence of the inventive composition preferably encodes a secreted protein, e.g., a protein that is naturally secreted by the infected cell. In contrast, the nucleic acid sequence can encode a protein that is not naturally secreted by the cell, but which is released by cell lysis induced by gene transfer vector (e.g., viral vector) infection. Alternatively, the nucleic acid sequence can encode a protein that is not naturally secreted by the cell (i.e., a non-secretable protein), but which comprises a signal peptide that facilitates protein secretion. In this manner, for example, the nucleic acid sequence encodes an endoplasmic reticulum (ER) localization signal peptide and the non-secretable protein. The ER localization signal peptide functions to direct DNA, RNA, and/or a protein to the membrane of the endoplasmic reticulum, wherein a protein is expressed and targeted for secretion. The ER localization signal peptide desirably functions to increase the secretion (i.e., the secretion potential) by a cell of (i) proteins that are not normally secreted (i.e., secretable) by the cell and/or (ii) proteins that are normally secreted by a cell, but in low (i.e., less than desired) quantities. The ER localization signal peptide encoded by the polynucleotide can be any suitable ER localization signal peptide or polypeptide (i.e., protein). For example, the ER localization signal peptide encoded by the nucleic acid sequence can be a peptide or polypeptide (i.e., protein) selected from the group consisting of nerve growth factor (NGF), immunoglobulin (Ig) (e.g., an Ig K chain leader sequence), and midkine (MK), or a portion thereof. Suitable ER localization signal peptides also include those described in Ladunga, Current Opinions in Biotechnology, 11, 13-18 (2000).

Although the nucleic acid sequence can encode any protein, the protein preferably is a secreted protein and is a tumor necrosis factor (TNF), a vascular endothelial growth factor (VEGF), or a pigment epithelium-derived factor (PEDF). Preferably, the gene transfer vector comprises a nucleic acid sequence coding for a TNF. Nucleic acid sequences encoding a TNF include nucleic acid sequences encoding any member of the TNF family of proteins (e.g., CD40 ligand and Fas ligand). The gene transfer vector preferably comprises a nucleic acid sequence coding for TNF-α. A nucleic acid sequence coding for TNF is described in detail in U.S. Pat. No. 4,879,226. Alternatively, the nucleic acid sequence can encode a VEGF. The nucleic acid sequence can encode any suitable VEGF isoform, including, but not limited to, VEGF₁₂₁, VEGF₁₄₅, VEGF₁₆₅, VEGF₁₈₉, or VEGF₂₀₆, which are variously described in U.S. Pat. Nos. 5,332,671, 5,240,848, and 5,219,739. Most preferably, because of their higher biological activity, the nucleic acid sequence encodes VEGF₁₂₁ or VEGF₁₆₅, particularly VEGF₁₂₁. A notable difference between VEGF₁₂₁ and VEGF₁₆₅ is that VEGF₁₂₁ does not bind to heparin with a high degree of affinity, as does VEGF₁₆₅. Other suitable VEGF peptides are VEGF-II, VEGF-C, and the like. The nucleic acid sequence also can encode a PEDF. PEDF, also known as early population doubling factor-1 (EPC-1), is a secreted protein having homology to a family of serine protease inhibitors named serpins. PEDF is made predominantly by retinal pigment epithelial cells and is detectable in most tissues and cell types of the body. PEDF has both neurotrophic and anti-angiogenic properties and, therefore, is useful in the treatment and study of a broad array of diseases. Nucleic acid sequences encoding anti-angiogenic derivatives of PEDF, known as SLED proteins (see, e.g., WO 99/04806), also can be used in connection with the invention. PEDF is further characterized in International Patent Applications WO 93/24529 and WO 99/04806, and the nucleic acid sequence encoding PEDF is described in U.S. Pat. No. 5,840,686 (Chader et al.).

The nucleic acid sequence can encode any variant, homolog, or functional portion of the aforementioned proteins. A variant of the protein can include one or more mutations (e.g., point mutations, deletions, insertions, etc.) from a corresponding naturally occurring protein. By “naturally occurring” is meant that the protein can be found in nature and has not been synthetically modified. Thus, where mutations are introduced in the nucleic acid sequence encoding the protein, such mutations desirably will effect a substitution in the encoded protein whereby codons encoding positively-charged residues (H, K, and R) are substituted with codons encoding positively-charged residues, codons encoding negatively-charged residues (D and E) are substituted with codons encoding negatively-charged residues, codons encoding neutral polar residues (C, G, N, Q, S, T, and Y) are substituted with codons encoding neutral polar residues, and codons encoding neutral non-polar residues (A, F, I, L, M, P, V, and W) are substituted with codons encoding neutral non-polar residues. In addition, a homolog of the protein can be any peptide, polypeptide, or portion thereof, that is more than about 70% identical (preferably more than about 80% identical, more preferably more than about 90% identical, and most preferably more than about 95% identical) to the protein at the amino acid level. The degree of amino acid identity can be determined using any method known in the art, such as the BLAST sequence database. Furthermore, a homolog of the protein can be any peptide, polypeptide, or portion thereof, which hybridizes to the protein under at least moderate, preferably high, stringency conditions. Exemplary moderate stringency conditions include overnight incubation at 37° C in a solution comprising 20% formamide, 5× SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1× SSC at about 37-50° C., or substantially similar conditions, e.g., the moderately stringent conditions described in Sambrook et al., supra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride, 75 mM sodium citrate at 42° C., or (3) employ 50% formamide, 5× SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at (i) 42° C. in 0.2× SSC, (ii) at 55° C. in 50% formamide and (iii) at 55° C. in 0.1× SSC (preferably in combination with EDTA). Additional details and an explanation of stringency of hybridization reactions are provided in, e.g., Ausubel et al., supra. A “functional portion” is any portion of a protein that retains the biological activity of the naturally occurring, full-length protein at measurable levels. A functional portion of the protein produced by expression of the nucleic acid sequence of the gene transfer vector can be identified using standard molecular biology and cell culture techniques, such as assaying the biological activity of the protein portion in human cells transiently transfected with a nucleic acid sequence encoding the protein portion.

The expression of the nucleic acid sequence encoding the protein is controlled by a suitable expression control sequence operably linked to the nucleic acid sequence. An “expression control sequence” is any nucleic acid sequence that promotes, enhances, or controls expression (typically and preferably transcription) of another nucleic acid sequence. Suitable expression control sequences include constitutive promoters, inducible promoters, repressible promoters, and enhancers. The nucleic acid sequence encoding the protein can be regulated by its endogenous promoter or, preferably, by a non-native promoter sequence. Examples of suitable non-native promoters include the cytomegalovirus (CMV) promoters, such as the CMV immediate-early promoter (described in, for example, U.S. Pat. No. 5,168,062), promoters derived from human immunodeficiency virus (HIV), such as the HIV long terminal repeat promoter, the phosphoglycerate kinase (PGK) promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad. Sci., 78, 144-145 (1981)), promoters derived from SV40 or Epstein Barr virus, an adeno-associated viral promoter, such as the p5 promoter, the sheep metallothionien promoter, the human ubiquitin C promoter, and the like. Alternatively, expression of the nucleic acid sequence encoding the protein can be controlled by a chimeric promoter sequence. The promoter sequence is “chimeric” in that it comprises at least two nucleic acid sequence portions obtained from, derived from, or based upon at least two different sources (e.g., two different regions of an organism's genome, two different organisms, or an organism combined with a synthetic sequence). Techniques for operably linking sequences together are well known in the art.

The promoter can be an inducible promoter, i.e., a promoter that is up- and/or down-regulated in response to an appropriate signal. For example, an expression control sequence up-regulated by a chemotherapeutic agent is particularly useful in cancer applications. The nucleic acid sequence preferably is operably linked to a radiation-inducible promoter, especially when the nucleic acid sequences encodes a TNF. The use of a radiation-inducible promoter provides control over transcription of the nucleic acid sequence, for example, by the administration of radiation to a cell or host comprising the gene transfer vector. Any suitable radiation-inducible promoter can be used in conjunction with the invention. The radiation-inducible promoter preferably is the early growth region-1 (Egr-1) promoter, specifically the CArG domain of the Egr-1 promoter. The Egr-1 promoter is described in detail in U.S. Pat. No. 5,206,152 and International Patent Application WO 94/06916. The promoter can be introduced into the genome of the gene transfer vector by methods known in the art, for example, by the introduction of a unique restriction site at a given region of the genome. Alternatively, the promoter can be inserted as part of the expression cassette comprising the nucleic acid sequence coding for the protein, such as a TNF.

Preferably, the nucleic acid sequence encoding the protein further comprises a transcription-terminating region such as a polyadenylation sequence located 3′ of the region encoding the protein. Any suitable polyadenylation sequence can be used, including a synthetic optimized sequence, as well as the polyadenylation sequence of BGH (Bovine Growth Hormone), polyoma virus, TK (Thymidine Kinase), EBV (Epstein Barr Virus), and the papillomaviruses, including human papillomaviruses and BPV (Bovine Papilloma Virus). A preferred polyadenylation sequence is the SV40 (human Sarcoma Virus-40) polyadenylation sequence.

In addition to the nucleic acid encoding the protein, the gene transfer vector can comprise at least one additional nucleic acid sequence encoding at least one other gene product, e.g., which itself performs a prophylactic or therapeutic function, or augments or enhances a prophylactic or therapeutic potential of the protein. The gene product encoded by the additional nucleic acid sequence can be an RNA, peptide, or polypeptide with a desired activity. If the additional nucleic acid sequence confers a prophylactic or therapeutic benefit, the nucleic acid sequence can exert its effect at the level of RNA or protein. Alternatively, the additional nucleic acid sequence can encode an antisense molecule, a ribozyme, a protein that affects splicing or 3′ processing (e.g., polyadenylation), or a protein that affects the level of expression of another gene within the cell (i.e., where gene expression is broadly considered to include all steps from initiation of transcription through production of a process protein), such as by mediating an altered rate of mRNA accumulation or transport or an alteration in post-transcriptional regulation. The additional nucleic acid sequence can encode any one of a variety of gene products that confers a prophylactic or therapeutic benefit, depending on the intended end-use of the composition. If, for example, the protein produced by expression of the nucleic acid sequence of the gene transfer vector induces killing of cancer cells, the additional nucleic acid can encode a protein that protects normal cells from the cytotoxic effects of the protein produced by expression of the nucleic acid sequence of the gene transfer vector. Alternatively, the additional nucleic acid can encode a protein that inhibits angiogenesis at the tumor site. Furthermore, the additional nucleic acid sequence can encode a gene product that does not function in a disease-specific manner. In other words, for example, the gene product may induce persistent expression of the nucleic acid sequence encoding the protein of the gene transfer vector. The additional nucleic acid sequence also can encode a factor that acts upon a different target than the protein encoded by the nucleic acid sequence of the gene transfer vector, thereby providing multifactorial treatment. The additional nucleic acid sequence can encode a chimeric protein for combination therapy. The additional gene product can be secreted, or remain within the cell in which it is produced unless or until the cell is lysed. A variety of gene products can enhance the therapeutic potential of the gene transfer vector in treating a specific disease.

The additional nucleic acid sequence can encode one gene product or multiple gene products. Alternatively, multiple additional nucleic acid sequences, each encoding one or more gene products, can be inserted into the gene transfer vector. In either case, expression of the gene product(s) can be separately regulated by individual expression control sequences, or coordinately regulated by one common expression control sequence. Alternatively, expression of the additional nucleic acid(s) can be regulated by the same expression control sequence that regulates expression of the protein encoded by the nucleic acid sequence of the gene transfer vector; however, any transcription terminating regions present in the nucleic acid encoding the protein would be eliminated to allow for transcriptional read-through of the additional nucleic acid sequence(s). The additional nucleic acid sequence(s) can comprise any suitable expression control sequence(s) and any suitable transcription-termination region(s) discussed herein in connection with expression of the protein produced by expression of the nucleic acid sequence of the gene transfer vector.

The composition comprises about 1×10⁵ or more particle units (pu) of the gene transfer vector. A “particle unit” is a single vector particle. The composition desirably comprises about 1×10⁶ particle units of the gene transfer vector (e.g., about 1×10⁷ or more particle units, about 1×10⁸ or more particle units, and about 1×10⁹ or more particle units). Preferably, the composition comprises about 1×10¹⁰ or more pu, 1×10¹¹ or more pu, 1×10¹² or more pu, 1×10¹³ or more pu, 1×10¹⁴ or more pu, or 1×10¹⁵ or more pu of the gene transfer vector, especially of a viral vector, such as a replication-deficient adenoviral vector. The number of particle units of the gene transfer vector in the composition can be determined using any suitable method known, such as by comparing the absorbance of the composition with the absorbance of a standard solution of gene transfer vector (i.e., a solution of known gene transfer vector concentration) as described further herein.

The carrier of the composition comprising the gene transfer vector can be any suitable carrier for the gene transfer vector. Suitable carriers for the gene transfer vector composition are described in U.S. Pat. No. 6,225,289. The carrier typically will be liquid, but also can be solid, or a combination of liquid and solid components. The carrier desirably is a pharmaceutically acceptable (e.g., a physiologically or pharmacologically acceptable) carrier (e.g., excipient or diluent). Pharmaceutically acceptable carriers are well known and are readily available. The choice of carrier will be determined, at least in part, by the particular gene transfer vector and the particular method used to administer the composition. The composition can further comprise any other suitable components, especially for enhancing the stability of the composition and/or its end-use. Accordingly, there is a wide variety of suitable formulations of the composition of the invention. The following formulations and methods are merely exemplary and are in no way limiting.

Formulations suitable for oral administration include (a) liquid solutions, such as an effective amount of the active ingredient dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base (such as gelatin and glycerin, or sucrose and acacia), and emulsions, gels, and the like containing, in addition to the active ingredient, such excipients as are known in the art.

Formulations suitable for administration via inhalation include aerosol formulations. The aerosol formulations can be placed into pressurized acceptable propellants, such as dichlorodifluoromethane, propane, nitrogen, and the like. They also can be formulated as non-pressurized preparations, for delivery from a nebulizer or an atomizer.

Formulations suitable for parenteral administration include aqueous and nonaqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described.

Formulations suitable for anal administration can be prepared as suppositories by mixing the active ingredient with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the gene transfer vector and physiological distress. Immune system suppressors can be administered with the composition method to reduce any immune response to the gene transfer vector itself or associated with a disorder. Alternatively, immune enhancers can be included in the composition to upregulate the body's natural defenses against disease. Moreover, cytokines can be administered with the composition to attract immune effector cells to the tumor site.

Anti-angiogenic factors, such as soluble growth factor receptors, growth factor antagonists, i.e., angiotensin, and the like, also can be part of the composition. Similarly, vitamins and minerals, anti-oxidants, and micronutrients can be co-administered with the composition. Antibiotics, i.e., microbicides and fungicides, can be present to reduce the risk of infection associated with gene transfer procedures and other disorders.

The ratio of the gene transfer vector to the protein in the inventive composition is about 6.4×10⁹ or more particle units of gene transfer vector:1 picogram of protein. The ratio of the amount of gene transfer vector (pu) to protein (pg) desirably is about 7×10⁹ or more pu:1 pg (e.g., about 7.5×10⁹ pu or more:1 pg, about 8×10⁹ pu or more:1 pg, about 8.5×10⁹ pu or more:1 pg, or even about 9.0×10⁹ pu or more:1 pg). The gene transfer vector to protein ratio preferably is about 1×10¹⁰ pu or more:1 pg (e.g., about 3×10¹⁰ pu or more:1 pg, about 5×10¹⁰ pu or more:1 pg, about 7×10¹⁰ pu or more:1 pg, or even about 9×10¹¹ or more pu:1 pg). The gene transfer vector to protein ratio more preferably is about 1×10¹¹ or more pu:1 pg (e.g., about 3×10¹¹pu or more:1 pg, about 5×10¹¹ pu or more:1 pg, about 7×10¹¹ or more pu:1 pg, or even about 9×10¹¹ or more pu:1 pg). Most preferably, the gene transfer vector to protein ratio is about 1×10¹² or more pu:1 pg (e.g., about 1×10¹³ or more pu:1 pg, about 1×10¹⁴ or more pu:1 pg, or even about 1×10¹⁵ or more pu:1 pg). Moreover, it is conceivable that the inventive composition comprises a highly purified gene transfer vector that lacks any protein produced through expression of the nucleic acid sequence of the gene transfer vector. In such cases, the gene transfer vector to protein ratio would be infinity, and the invention also encompasses compositions of such high purity. For the purposes of considering the ratio in terms of particle units when the gene transfer vector is a viral vector, it can be assumed that there are 100 particles/plaque forming unit (pfu) (e.g., 1×10¹² pfu is equivalent to 1×10¹⁴ pu).

The ratio of gene transfer vector to protein in the composition can be determined by any suitable method, for example, the number of gene transfer vector particles in the composition can be quantified by absorbance techniques. The absorbance of a gene transfer vector composition sample is determined using methods known in the art (see, e.g., Mittereder et al., J. Virol., 70, 7498-7509 (1996)). The absorbance of a standard gene transfer vector composition, i.e., a solution of gene transfer vector of known concentration, is similarly determined. Through a comparison of the absorbance of the sample solution and the absorbance of the standard solution, the concentration of gene transfer vector, e.g., the number of replication-deficient adenoviral particles in a given volume, in the sample solution is determined.

The standard absorbance can be a single standard absorbance or a series or group of standard absorbances indicative of a range of concentrations of the gene transfer vector in the composition. The sample and standard absorbances can be presented in similar or different (though preferably similar) formats, measurements, or units as long as a useful comparison can be performed. For example, a suitable standard absorbance can be an absorbance that is determined from a standard solution of replication-deficient adenoviral vector that has been treated in the same manner as a sample solution of replication-deficient adenoviral vector has been treated in accordance with the methods described herein.

Quantification of the number of gene transfer vector particles is accomplished by comparing the sample absorbance to the standard absorbance in any suitable manner. For example, sample absorbance and standard absorbance can be compared by calculating a standard curve of the area under the peak corresponding to the gene transfer vector elution from the chromatography resin on an absorbance versus time chromatograph. The absorbance of different known concentrations of gene transfer vector can be plotted on a graph, creating a standard curve. Using linear regression analysis, the sample concentration then can be determined.

In order to determine the ratio of the amounts of gene transfer vector to protein in the inventive composition, the mass of the protein produced by expression of the nucleic acid sequence of the gene transfer vector in the composition is quantified. Such quantification can be carried out using any suitable method of protein quantification. Suitable methods of protein quantification include Western blot, enzyme-linked immunosorbent assay (ELISA), the BCA assay (Smith et al., Anal. Biochem., 150,76-85 (1985)), the Lowry protein assay (described in, e.g., Lowry et al., J. Biol. Chem., 193, 265-275 (1951)), which is a calorimetric assay based on protein-copper complexes, and the Bradford protein assay (described in, e.g., Bradford et al., Anal. Biochem., 72, 248 (1976)), which depends upon the change in absorbance in Coomassie Blue G-250 upon protein binding. When the protein is TNF-α, the concentration of the protein in the composition is preferably determined by an ELISA assay specific for human TNF-α(R&D Systems, Inc., Minneapolis, Minn.); however the ordinarily skilled artisan will appreciate that any art-recognized method for detecting and quantifying proteins in solution may be used in connection with the invention.

Once the amounts of gene transfer vector and protein in the composition have been established, the ratio between these amounts can be calculated. One of ordinary skill in the art will recognize that calculating ratios in general requires only basic arithmetic techniques. The ratio is calculated by dividing the amount of gene transfer vector in particle units in the composition by the amount of protein produced by expression of the nucleic acid sequence of the gene transfer vector in picograms in the same composition. The result of this calculation is normalized such that a ratio is expressed in terms of particle units of gene transfer vector to one picogram of protein. Alternatively, the individual concentration measurements of gene transfer vector and protein can be separately normalized to the total composition volume (e.g., pu/ml or pg/ml) prior to dividing the gene transfer vector concentration by the protein concentration. Such a ratio results in a ratio expressed in terms of particle units (pu) of gene transfer vector to picograms (pg) of protein (i.e., pu:pg), which can be normalized to a ratio of gene transfer vector particles units per one picogram of protein.

Gene transfer vector purification to enhance the concentration of the gene transfer vector in the composition can be accomplished by any suitable method, such as by density gradient purification (e.g., cesium chloride (CsCl)) or by chromatography techniques (e.g., column or batch chromatography). For example, the gene transfer vector composition can be subjected to two or preferably three CsCl density gradient purification steps. The gene transfer vector, preferably a replication-deficient adenoviral vector, is desirably purified from cells infected with the replication-deficient adenoviral vector using a method that comprises lysing cells infected with adenovirus, applying the lysate to a chromatography resin, eluting the adenovirus from the chromatography resin, and collecting a fraction containing adenovirus.

The cells can be lysed using any suitable method, such as exposure to detergents, freeze-thawing, and cell membrane rupture (e.g., via French press or microfluidization). The cell lysate then optionally can be clarified to remove large pieces of cell debris using any suitable method, such as gentle centrifugation, filtration, or tangential flow filtration (TFF). The clarified cell lysate then optionally can be treated with an enzyme capable of digesting DNA and RNA (a “DNase/RNase”) to remove any DNA or RNA in the clarified cell lysate not contained within the gene transfer vector particles.

Once the cell lysate is clarified, it optionally can be chromatographed on an anion exchange pre-resin prior to purification. Any suitable anion exchange chromatography resin can be used in the pre-resin. A desirable pre-resin anion exchange chromatography resin in the context of the invention is Q Ceramic HyperD™ F, commercially available from BioSepra, Villeneuve-La-Garenne, France. The cell lysate is eluted from the anion exchange pre-resin chromatography resin in any suitable eluant (e.g., 600 mM NaCl). Following chromatography on the pre-resin, the gene transfer vector is purified from the cell lysate by purification chromatography. Any suitable purification chromatography resin can be used to purify the gene transfer vector from the cell lysate. Preferably, the purification chromatography resin is an anion exchange chromatography resin. The anion exchange chromatography resin desirably has a surface group selected from the group consisting of dimethylaminopropyl, dimethylaminobutyl, dimethylaminoisobutyl, and dimethylaminopentyl, especially when the gene transfer vector is an adenoviral vector. The surface group preferably is dimethylaminopropyl. The surface group can be linked to a matrix support through any suitable linker group as is known in the art. Sulphonamide and acrylate linkers are among those suitable in the context of the invention. The matrix support can be composed of any suitable material; however, it is preferable for the matrix support to be a perfusive anion exchange chromatography resin such that intraparticle mass transport is optimized.

The anion exchange chromatography resin is preferably perfusive, comprising large (e.g., 6,000-8,000 Å diameter) pores that transect the particles, as well as a network of smaller pores which supplement the surface area of the large diameter pores. Such perfusive chromatography resins are well-known in the art and, for example, are more fully described in Afeyan et al., J. Chromatogr., 519, 1-29 (1990), and U.S. Pat. Nos. 5,384,042, 5,228,989, 5,552,041, 5,605,623, and 5,019,270. A suitable perfusive anion exchange chromatography resin is POROS® 50D resin (commercially available from Applied Biosystems, Inc., Foster City, Calif.). Anion exchange chromatography resins can be used either in a “batch” configuration or, preferably, in a “flow-through” or “continuous” configuration, especially in the form of a column. Desirably, the gene transfer vector can be further purified by chromatography on a size exclusion column containing Sepharose® 4 Fast Flow chromatography medium equilibrated with final formulation buffer (FFB), and subsequent filtration.

A replication-deficient adenoviral vector purified in accordance with the above-described method does not have a substantially lower particle unit to plaque forming unit (pfu) ratio (pu/pfu) than a CsCl density gradient-purified adenovirus. That is, the pu/pfu of the purified replication-deficient adenoviral vector is at least 50% that of the CsCl density gradient-purified adenovirus, preferably at least about 85% that of the CsCl density gradient-purified adenovirus, and more preferably at least about 95% that of the CsCl density gradient-purified adenovirus. Moreover, the purity of the chromatographed replication-deficient adenoviral vector composition preferably is substantially at least that of an identical solution of replication-deficient adenoviral vector that is subjected to standard triple CsCl density gradient purification (i.e., is as substantially pure as triple CsCl density gradient-purified adenovirus, e.g., is at least 90% as pure, preferably is at least 97% as pure, more preferably is at least 99% as pure as triple CsCl gradient-purified adenovirus, and most preferably is at least 150% as pure as a triple CsCl gradient-purified adenovirus).

Following application of the cell lysate to the chromatography resin, the gene transfer vector is eluted from the resin using a suitable eluant. Suitable eluants are typically ionic in character and preferably include sodium chloride in a buffered solution. The eluant can be applied to the chromatography resin in a discontinuous or continuous gradient and at high concentrations (e.g., at least about 75% of the concentration that is necessary to elute the gene transfer vector from the chromatography resin, preferably between about 85% to about 90% of the concentration that is necessary to elute the gene transfer vector from the chromatography resin), while elution of the gene transfer vector can occur at any suitable flow rate (e.g., from about 100 cm/hr to about 1,500 cm/hr, preferably from about 500 cm/hr to about 1,250 cm/hr).

When the protein of the invention is an antitumor or anticancer agent, especially a TNF, the invention further provides a method of treating a tumor or cancer in a host comprising administering the inventive composition to a host in need thereof. When the protein is a VEGF, the invention provides a method of treating coronary artery disease, peripheral vascular disease, congestive heart failure (e.g., left ventricular dysfunction and left ventricular hypertrophy), neuropathy (peripheral or otherwise), avascular necrosis (e.g., bone or dental necrosis), mesenteric ischemia, impotence (or erectile dysfunction), incontinence, arterio-venous fistula, veno-venous fistula, stroke, cerebrovascular ischemia, muscle wasting, pulmonary hypertension, gastrointestinal ulcers, vasculitis, non-healing ischemic ulcers, retinopathies, restenosis, cancer, and radiation-induced tissue injury (such as that common with cancer treatment), as well as assisting with wound healing (e.g., healing of ischemic ulcers), plastic surgery procedures (e.g., healing or reattachment of skin and/or muscle flaps), bone healing, ligament and tendon healing, spinal cord healing and protection, prosthetic implant healing, vascular graft patency, and transplant longevity, in a host comprising administering the inventive composition to a host in need thereof. Similarly, when the protein is a PEDF the invention provides a method of treating ocular-related disorders associated with impaired vasculature of the eye in a host comprising administering the inventive composition to a host in need thereof. Examples of such ocular-related disorders include age related macular degeneration, diabetic retinopathy, corneal neovascularization, choroidal neovascularization, neovascular glaucoma, cyclitis, Hippel-Lindau Disease, retinopathy of prematurity, and the like.

One skilled in the art will appreciate that suitable methods of administering the composition of the invention to an animal (especially a human) for therapeutic or prophylactic purposes, e.g., gene therapy, vaccination, and the like (see, for example, Rosenfeld et al., Science, 252, 431-434 (1991), Jaffe et al., Clin. Res., 39(2), 302A (1991), Rosenfeld et al., Clin. Res., 39(2), 311A (1991), Berkner, BioTechniques, 6, 616-629 (1988)), are available, and, although more than one route can be used to administer the composition, a particular route can provide a more immediate and more effective reaction than another route. The dose administered to an animal, particularly a human, in the context of the invention will vary with the particular gene transfer vector, the composition containing the gene transfer vector and the carrier therefor (as discussed above), the method of administration, and the particular site and organism being treated. The dose should be sufficient to effect a desirable response, e.g., therapeutic or prophylactic response, within a desirable time frame. Thus, the dose of the gene transfer vector of the inventive composition typically will be about 1×10⁵ or more particle units (e.g., about 1×10⁶ or more particle units, about 1×10⁷ or more particle units, 1×10⁸ or more particle units, 1×10⁹ or more particle units, 1×10¹⁰ or more particle units, 1×10¹¹ or more particle units, or about 1×10¹² or more particle units). The dose of the gene transfer vector typically will not be 1×10¹³ or less particle units (e.g., 4×10¹² or less particle units, 1×10¹² or less particle units, 1×10¹¹ or less particle units, or even 1×10¹⁰ or less particle units).

The inventive method of treating a disease or disorder, particularly a tumor or cancer, in a host further can comprise the administration (i.e., pre-administration, coadministration, and/or post-administration) of other treatments and/or agents to modify (e.g., enhance) the effectiveness of the method. For example, an adenoviral vector comprising a nucleic acid sequence coding for a TNF that is operably linked to a radiation-inducible promoter can be administered in conjunction with the administration of radiation. The radiation can be administered to a host in any suitable manner, for example, by exposure of a host to an external source of radiation (e.g., infrared radiation), or through the use of an internal source of radiation (e.g., through the chemical or surgical administration of a source of radiation). For instance, the aforementioned adenoviral vector can be used in conjunction with brachytherapy, wherein a radioactive source is placed (i.e., implanted) in or near a tumor to deliver a high, localized dose of radiation. Radiation is desirably administered in a dose sufficient to induce the expression of the nucleic acid sequence encoding the TNF to produce a therapeutic level of the TNF in the host. The total dose of radiation administered to the host preferably is at least about 30 gray (Gy) to about 70 Gy (e.g., about 40 Gy or more, about 50 Gy or more, or about 60 Gy or more). Although administration of radiation post-administration of the inventive composition is the preferred method by which therapeutic levels of the TNF are induced, pre- and/or co-administration of radiation (or any other agent), alone or in addition to post-administration of radiation, also is within the scope of the invention. In such cases, radiation can be administered as an adjuvant therapy to increase the likelihood of killing the maximum number of tumor or cancer cells.

The method of the invention, additionally or alternatively to the administration of radiation, further can comprise the administration of other substances which locally or systemically alter (i.e., diminish or enhance) the effect of the composition on a host. For example, substances that diminish any systemic effect of the protein produced through expression of the nucleic acid sequence of the gene transfer vector in a host can be used to control the level of systemic toxicity in the host. Likewise, substances that enhance the local effect of the protein produced through expression of the nucleic acid sequence of the gene transfer vector in a host can be used to reduce the level of the protein required to produce a prophylactic or therapeutic effect in the host. Such substances include antagonists, for example, soluble receptors or antibodies directed against the protein produced through expression of the nucleic acid sequence of the gene transfer vector, and agonists of the protein. Suitable antagonists, agonists, and other substances that alter the effects of proteins, particularly secreted proteins such as TNF, VEGF, and PEDF, are available and generally known in the art.

The following examples further illustrate the invention but, of course, should not be construed as in any way limiting its scope.

### EXAMPLE 1

This example describes the formulation of a composition comprising a replication-deficient adenoviral vector comprising a nucleic acid sequence encoding a TNF.

An expression cassette comprising the human TNF-α gene under the control of the Egr-1 promoter was inserted into the E1 region of a replication-deficient adenoviral vector containing deletions in the E1, E3, and E4 regions of the adenoviral genome. The resulting AdTNF vector was propagated in 293-ORF6 cells (derived from 293 human embryonic kidney cells), which compliment for E1 and E4 deficiencies, in the presence or absence of serum in the growth medium. The cells then were lysed, clarified, and concentrated according to methods known in the art. After 24 hours, the crude concentrate of the AdTNF vector was applied to a Q Ceramic HyperD™ F capture column and eluted with a step gradient of 360 to 475 mM NaCl. The material eluted from the column was diluted to adjust for conductivity and pH and was loaded onto an equilibrated ion exchange purification column containing POROS® 50D resin. Purified AdTNF vector was eluted from the column in a concentration of 450 mM NaCl. The material collected from the purification column was loaded onto a size exclusion column containing Sepharose® 4 Fast Flow chromatography medium which was equilibrated with final formulation buffer (FFB). The bulk product then was filtered through a 0.2 μm pre-sterilized filter, and the collected material was frozen at −70° C. to −85° C. until formulation. The AdTNF vector was formulated for human administration by diluting the AdTNF vector bulk product in a carbohydrate-based stabilizing buffer (Chesapeake Biologics Laboratory, Inc., Baltimore, Md.) to form a total dose of at least 4×10⁹ pu of the AdTNF vector in a total volume of 2-8 ml of carrier.

### EXAMPLE 2

This example demonstrates that the composition of Example 1 comprises a relatively high quantity of particle units of the AdTNF vector and a relatively high ratio of the AdTNF vector particle units to TNF protein in the composition.

Following capture column chromatography of AdTNF in Example 1, the concentration of the TNF-α protein in the eluted composition is quantified using an ELISA assay specific for human TNF-α (R&D Systems, Inc., Minneapolis, Minn.). In particular, samples collected from the capture column and standard samples are applied to a microplate that has been pre-coated with an anti-TNF-α monoclonal antibody. All unbound substances are washed away, and a horseradish peroxidase-linked polyclonal antibody specific for TNF-α is added to the microplate. Following a final wash step, the substrate (hydrogen peroxide) is added to the wells, and color develops in proportion to the amount of TNF-α bound in the first step. The amount of TNF-α is measured by determining the optical density of the sample at 450 nm. Using this method, the amount of the TNF-α protein in the eluted composition, i.e., after capture column chromatography of the AdTNF vector crude concentrate, is approximately 15.6 picograms per milliliter (pg/nl). TNF-α protein levels are virtually undetectable following both purification and size exclusion column chromatography (lower limit of detection claimed by manufacturer is less than 4.4 pg/ml).

The number of AdTNF vector particles in the composition is measured following the size exclusion chromatography described in Example 1. The absorbance of a sample of the composition eluted from the chromatography resin is determined using methods known in the art (see, e.g., Mittereder et al., supra). For comparison, the absorbance of a standard solution of adenovirus, i.e., a solution of adenovirus of known concentration, is determined. Through a comparison of the absorbance of the sample solution and the absorbance of the standard solution, the concentration of replication-deficient adenoviral particles, i.e., the number of replication-deficient adenoviral particles, and, therefore, of the AdTNF vector particles, in a given volume of the sample solution is determined to be 1×10¹¹ pu/ml.

Accordingly, the ratio of AdTNF vector:TNF protein, calculated by dividing the concentration of the AdTNF vector particles (1×10¹¹ pu/ml) by the TNF protein concentration (<4.4 pg/ml) in the composition, is determined to be at least 22.7×10⁹ pu AdTNF vector:1 pg TNF protein.

### EXAMPLE 3

This example demonstrates the use of the AdTNF vector composition to treat a tumor or cancer in a host.

Three treatment groups were established, each comprising eight nude mice having radio-resistant human squamous tumor cell line (SQ-20B) xenograft tumors. The first treatment group received a dose of 5×10¹⁰ particle units (pu) of the AdTNF vector (in a total composition volume of 32 μl with a viral buffer) by direct intratumoral injection of the AdTNF vector composition (prepared as described in Example 1) at five sites (four injections around the periphery of each tumor and one injection into the center of each tumor) at days 0, 4, 7, and 11. The second treatment group received the same dose of the AdTNF vector administered in conjunction with exposure of the tumor to 5 Gy of radiation on days 0-4 and 7-9 (totaling 40 Gy of radiation). The third treatment group received only the radiation exposure to which the second treatment group was exposed, but no doses of the ADTNF vector.

At day 11, tumor necrosis and ulceration was visible in animals in the first and second treatment groups. At this time, one animal in the first treatment group and three animals in the second treatment group had no visible tumors. After 62 days, 100% (8/8) of the animals in the second treatment group were cured (i.e., no visible tumors were present), while 75% (6/8) of the animals in the first treatment group were cured, and only 14% (1/7) of the animals in the third treatment group (no vector) were cured.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition comprising (a) about 1×10⁵ or more particle units of a gene transfer vector comprising a nucleic acid sequence encoding a protein and (b) a carrier therefor, wherein the ratio of the gene transfer vector to the protein in the composition is about 6.4×10⁹ or more particle units gene transfer vector:1 picogram protein.

2. The composition of claim 1, wherein the protein is a tumor necrosis factor.

3. The composition of claim 1, wherein the protein is a vascular endothelial growth factor.

4. The composition of claim 1, wherein the protein is a pigment epithelium-derived factor.

5. The composition of claim 1, wherein the gene transfer vector is a viral vector.

6. The composition of claim 5, wherein the gene transfer vector is a replication-deficient adenoviral vector.

7. The composition of claim 6, wherein the replication-deficient adenoviral vector comprises an adenoviral genome deficient in at least one replication-essential gene function of one or more regions of the adenoviral genome.

8. The composition of claim 7, wherein the one or more regions of the adenoviral genome are selected from the group consisting of the E1, E2, and E4 regions.

9. The composition of claim 8, wherein the replication-deficient adenoviral vector comprises a deficiency in at least one replication-essential gene function of the E1 region.

10. The composition of claim 9, wherein the replication-deficient adenoviral vector comprises a deficiency in a replication-essential gene function in an adenoviral E1A region and a deficiency in a replication-essential gene function in an adenoviral E1B region.

11. The composition of claim 9, wherein the replication-deficient adenoviral vector further comprises a deficiency in at least one replication-essential gene function of the E4 region.

12. The composition of claim 9, wherein the nucleic acid sequence encoding the protein is located in the E1 region of the adenoviral genome.

13. The composition of claim 2, wherein the tumor necrosis factor is TNF-α.

14. The composition of claim 2, wherein the nucleic acid sequence encoding the tumor necrosis factor is operably linked to a radiation-inducible promoter.

15. The composition of claim 14, wherein the radiation-inducible promoter is the Egr-1 promoter.

16. The composition of claim 3, wherein the vascular endothelial growth factor is VEGF₁₂₁.

17. The composition of claim 6, wherein the composition comprises about 1×10⁶ to about 1×10¹³ particle units of the replication-deficient adenoviral vector.

18. A method of treating a tumor or cancer in a host comprising administering the composition of claim 2 to a host in need thereof.

19. The method of claim 18, further comprising the administration of radiation to the host.

20. The method of claim 19, wherein the radiation induces expression of the nucleic acid sequence encoding the tumor necrosis factor to produce the tumor necrosis factor in the host.
